(19) United States
(12) Patent Application Publication
(10) Pub. No.: US 2018/0151259 A1
(43) Pub. Date: May 31, 2018

(54) METHOD AND SYSTEM FOR SAFELY GUIDING INTERVENTIONS IN PROCEDURES THE SUBSTRATE OF WHICH IS THE NEURONAL PLASTICITY

(71) Applicant: FUNDACIÓ INSTITUT GUTTMANN, BADALONA (Barcelona) (ES)

(72) Inventors: José Maria TORMOS MUÑOZ, Valencia (ES); Alejandro GARCÍA RUDOLPH, Barcelona (ES); Eloy OPISSO SALLERA, Barcelona (ES); Maria Teresa ROIG ROVIRA, Barcelona (ES); Alberto GARCÍA MOLINA, Barcelona (ES)

(73) Assignee: FUNDACIÓ INSTITUT GUTTMANN, BADALONA (Barcelona) (ES)

(21) Appl. No.: 15/880,737

(22) Filed: Jan. 26, 2018

Related U.S. Application Data
(63) Continuation-in-part of application No. 14/224,936, filed on Mar. 25, 2014, now abandoned.
Application No. 14/224,936 is a continuation-in-part of application No. 13/126,838, filed on May 16, 2011, now abandoned.

(51) Int. Cl. G16H 20/70; G09B 19/00; G09B 7/07; A61B 5/16; G06N 3/08; G09B 7/02

(52) U.S. Cl. CPC G16H 20/70; G09B 19/00; G09B 7/02; A61B 5/16; G06N 3/08; G09B 7/07

(57) ABSTRACT

A method, and system for implementing the method, that generates a database with information regarding users in relation to interventions to be performed and to the user responses to the performance thereof, and analyzing it to generate candidate predictions from which final or optimum predictions are determined. The generation of candidate predictions and the subsequent determination of final predictions is carried out by corresponding steps of classification at different levels based on heuristic rules. The method and the system are particularly applicable in processes such as those related to neurorehabilitation, neuroeducation/neurolearning or cognitive neurostimulation.

### REFERENCE TO COPENDING PATENT APPLICATIONS

This is a continuation-in-part of application Ser. No. 14/224,936 that was filed Mar. 25, 2014, now abandoned, which is in turn a continuation-in-part of application Ser. No. 13/126,838 that was filed May 16, 2011, now abandoned, which in turn is a national phase entry from PCT/ES2008/000677 that was filed Oct. 31, 2008, now expired.

### FIELD OF THE ART

The present invention relates to a method, and system for implementing the method, of safely guiding interventions in processes the substrate of which is the neuronal plasticity such as neurorehabilitation processes, neuroeducation/neurolearning processes or cognitive neurostimulation processes, by means of generating and using a database with information regarding a plurality of users, and particularly it relates to a method which comprises analyzing said database for generating candidate predictions, from which final or optimum predictions are determined, said generation of candidate predictions and said subsequent determination of final predictions been carried out by means of corresponding steps of classification at different levels.

### STATE OF THE ART

Different proposals regarding the supply or application to users or patients of interventions in processes the substrate of which is the neuronal plasticity such as those comprising cognitive tests or tasks, or of other type, and the subsequent evaluation of their responses, are known.

As described in “The Plastic Human Brain Cortex”, Annual Reviews Neuroscience 2005.28:377-401 of Alvaro Pascual-Leone, Amir Amedi, Felipe Fregni and Lotfi B. Merabet, plasticity is an intrinsic property of the nervous system consisting in the capacity for modifying its structure from experience. This property allows it to learn, to acquire new skills, or even to recover from the alterations caused by a lesion. However, the changes do not necessarily result in a benefit; occasionally, these changes can generate the onset of diseases or be responsible for making the alterations derived from a lesion chronic. There is a challenge to sufficiently learn about neuronal plasticity for modulating it and thus achieving the best response of the behaviour for a specific patient. In order to carry out said modulation, as indicated in the aforementioned article, different types of interventions can be carried out such as those based on conduct modification, or those including different invasive or non-invasive cortical stimulation techniques.

The manner that Neurobiological functions are performed is explained as follows.

The main goal of the nervous system is to regulate our interaction with the environment. This means to select the most optimal response to the changes in the external environment to maintain the homeostasis (balanced regulation of our internal systems). In this sense, any function of the nervous system can be considered as a sensory motor integration response, in which sensory stimuli (external and/or internal) are processed by elaborating motor response (at muscular-skeletal or vegetative level). Behavior can be described as the integration of multiple level of sensory motor integrations, with a hierarchical order, in which most new structures (from the phylogenetical point of view) play an inhibitory-regulatory control over most elementary levels of integration. Even though this integrative approach, psychology and cognitive sciences have devoted big efforts to identify more elementary components, that have been described as cognitive functions, that represent identifiable strategies to process specific types of stimuli and their corresponding motor responses. Founded on this statement, psychology, neuropsychology and cognitive neuroscience are based on specific paradigms that allow objectively exploring neurobiological functions by presenting specific sensory stimuli and objectively assessing the correspondent motor (behavioral) response. A more detailed explanation from this perspective can be found in several integrative theories as the Reticular Paradigm of Memory [24], the Mirror Neuron theory of action understanding [25] and, at more conceptual level, the Embodied Cognition theory [26].

The significance of objective indicators is explained as follows.

To assess neurobiological functions, psychology, neuropsychology and cognitive neuroscience, as scientific disciplines, have developed scientific validated instruments, which allows reproduce specific behavioral paradigms, associated to a numerical range or normative responses. These numerical measures have been accepted as objective indicators of cognitive functions and their neurobiological correlates, allowing for identification of normal and pathological indicators. In the same way, numerical values also allow for the identification of different degrees of impairment, as well as calculate improvement, impairment or lack of changes in successive measurements. To the main aim of the proposed method, among all the neurobiological functions it has been focused on a selection of functions and sub-functions: Attention (sub-functions); Memory (sub-functions); and Executive Functions (sub-functions).

Test procedures and validation procedure for validated psychometric tests are explained in the following paragraphs, which also refer to references by number the corresponds with the consecutive numbering of such references under a section entitled References for test procedures and validation procedure for validated psychometric tests.

Psychometric tests are defined as tests that measure a psychological construct, such as cognitive functioning. The assessment of cognitive abilities involves the use of standardized assessment tools. Before being considered suitable, such tools must offer accurate, valid and interpretable data for the population's assessment. Such measures are supposed to provide scientifically robust results. The performance of these measures comes from their reliability, stability, internal consistency, equivalence and validity, considered as the main instruments' measurement properties [1, 2].

Reliability is the ability to reproduce a consistent result in time and space, or from different observers. It is important to highlight that the reliability is not a fixed property of a test. On the contrary, reliability relies on the function of the test, of the population in which it is used, on the circumstances, on the context; that is, the same test may not be considered reliable under different conditions [3]. Three important reliability criteria, of great interest in this work are described below: (i) stability, (ii) internal consistency and (iii) equivalence. We will also describe the most used statistical methods to assess each of the aspects.

Stability measures how similar the results are when measured at two differe7nt times, [4] that is, it estimates the consistency of measurement repetition. Stability assessment is usually performed using test-retest method. The intraclass correlation coefficient (ICC) is one of the most used tests to estimate variables stability, because it takes into account the measurement errors [5]. The test-retest reliability tends to reduce when the test reapplication is extended. The time span between measurements will influence the interpretation of reliability in the test-retest; therefore, the time span from 10 to 14 days is considered adequate for the test and retest [6]. With regard to the sample, a number of at least 50 subjects is considered adequate [3].

Internal consistency—or homogeneity—shows if all subparts of an instrument measure the same characteristic [7]. An estimate of low internal consistency may indicate that the items measure different constructs or that the answers to the questions of the instrument are inconsistent. Most researchers assess internal consistency of instruments through Cronbach's alpha coefficient [8].

Cronbach's alpha coefficient demonstrates the covariance level between the items of a scale. Thus, the lower the sum of items variance is, the more consistent the instrument will be. Studies establish that values higher than 0.7 are ideal, besides, values under 0.70—but close to 0.60—are considered satisfactory [9]. For instruments whose variables are dichotomous, Kuder-Richardson is the most adequate test, not Cronbach's alpha. Just as in the interpretation of the coefficient's results, values close to 1.00 are considered ideal [10].

Equivalence is the concordance degree of two or more observers regarding an instrument scores. The most common way of assessing the equivalence is the inter-observer reliability, which involves the independent participation of two or more raters [11]. In this case, the instrument is filled in by the raters [12]. The inter-observer reliability depends mainly on an adequate training process of the raters and of standardization for the test application. When there is high concordance between the raters, it can be inferred that the measurement errors were minimized [13].

Kappa coefficient is a measure used to assess inter-observers, applied to category variables. It is a concordance measure between the raters and has a maximum value of 1.00. The higher the Kappa value is, the higher the concordance between the raters will be. Values close to or below 0.00 indicate lack of concordance [14].

Validity refers to the fact that a tool measures exactly what it proposes to measure [15]. Validity is not an instrument characteristic and must be determined regarding a specific matter, once it refers to a defined population. The measurement properties—validity and reliability—are not totally independent. Researchers affirm that an instrument that is not reliable cannot be valid; however, a reliable instrument, can, sometimes, be invalid [16]. Thus, a high reliability does not ensure an instrument validity. With regard to validity types, in this present work the three main ones are considered: (i) content validity, (ii) criterion validity and (iii) construct validity.

Content validity refers to the degree in which the instrument content adequately reflects the construct that is being measured, [17] that is, it evaluates how much an items sample represents in a defined universe or content domain. As there is no statistical test to assess specifically the content validity, usually researchers use a qualitative approach, through the assessment of an experts committee, [18] and then, a quantitative approach using the content validity index (CVI) [19]. The CVI measures the proportion or percentage of judges who agree on certain aspects of a tool and its items. This method consists of a four-point Likert scale, where: 1=non-equivalent item; 2=the item needs to be extensively revised so equivalence can be assessed; 3=equivalent item, needs minor adjustments; and 4=totally equivalent item. The items that receive 1 or 2 points have to be revised or removed. To calculate the CVI for each item of the instrument, you have to add all the answers 3 and 4 of the experts committee and divide the result by the number of answers [19]. The acceptable concordance index among the experts committee must be at least 0.80 and, preferably, higher than 0.90 [20].

Criterion validity Is the relation between the score of a certain instrument and some external criterion [21]. This criterion has to be a widely accepted measure, with the same characteristics of the assessment tool, that is, an instrument or criterion considered ‘gold standard’ [12]. In assessments of criterion validity, researchers test the validity of a measure comparing the measurement results with the ‘gold standard’ or established criterion. If the target test measures what is intended to be measured, then its results must agree with the results of the ‘gold standard’ or the criterion. Whatever the assessed construct is, it is considered valid when its scores correspond to the scores of the chosen criterion. The criterion validity may be checked by a correlation coefficient [13]. The scores of the measurement instrument are correlated with the scores of the external criterion and this coefficient is analyzed. Values close to 1.00 suggest correlation, whereas values close to 0.00 suggest there is no correlation. Correlation coefficients equal to 0.70 or over are desirable [13].

Construct validity is the degree to which a group of variables really represents the construct to be measured [22]. This type of validity is hardly obtained on a single study; usually, several researches are developed on the theory of the construct, which is intended to be measured [13]. It is essential that there is a theory associated to the process of construct validity. That way, the more evidences there are, and the more valid the results interpretation will be. A common technique used among researchers to assess the construct validity is the structural equation modeling (SEM) [22]. This method aims to explain the relations between multiple variables. A conventional model in SEM is, actually, formed by two models: the measurement model, which represents how the variables measured are unified to represent the construct; and the structural model, which demonstrates how the constructs are associated [23]. To assess the measurement model it is common to verify the convergent and discriminant validities. At convergent validity, the items that indicate a specific construct must have a high proportion of variance in common. And the discriminant validity it is the degree in which the construct differs from the others. After the assessment of the convergent and discriminant validities, the next step is to analyze the structural model or theoretical model. They are the conceptual representation of the relation between the constructs. To test the structural model, the researcher must focus on the general adjustment of the model and on the relation between the constructs. To verify the relations between constructs and the items of the model, the Student's t-test and chi-squared test are performed, in which it is possible to verify if the parameters are significantly different from zero.

The nature of data results and their significance is explained as follows.

As stated in the kernel of the proposed methodology, cognitive functions are the result of neurobiological functions, as the resultant of the synaptic transmission between a specific neural network. Based on this, neurobiological functions depend on the structural pathways defined by such neural network. An intrinsic characteristic of neural networks, defined as plasticity, is that the specific pattern of connections can be modified by (and depend on) the activation of the connections itself. Thus, connections that are more frequently used, trend to be reinforced, even can induce the formation of new connections to optimize the network, while connections that are less used tend to be diminished or abolished. Behavioral consequences of their activation also have an impact on reinforcement or weakness of connections. In general terms, successful responses would induce reinforcement while non-successful responses will not.

Because of this, interventions cannot be considered as a exposure to an event, but needs to be considered as a process itself, where any stimulus require an specific response of the subject to activate the neural network in an specific way. Consequences of any one of the responses will have greater impact on final outcome of the global intervention. Neural networks activated in right responses is different that the pattern of activation in wrong responses. This will have implications on the ability to consolidate new functions or learn new skills. “Learning substrates in the primate prefrontal cortex and striatum: sustained activity related to successful actions. Histed, M H, Pasupathy, A and Miller, E K. 2, 244-5, 2009, Neuron, Vol. 63”.

As stated in the proposed methodology, development, acquisition, modification or restoration of cognitive functions are subjected to plasticity, as an intrinsic property of the nervous system. This is a complex phenomenon, which has not been deeply understood yet. However, the present invention can monitor and guide it, trying to use strategic interventions to optimize behavioral outcome, as is the case of neuropsychological rehabilitation or cognitive training. To this aim, the method needs to objectively characterize cognitive (neurobiological) functions by using neuropsychological tests and batteries; to define normality or the level or impairment, based on reliable objective indicators; to classify the interventions based on their objective results; and identify strategies that lead to improved behavior from those strategies that do not. An ANNEX is provided that explains the neuropsychological tests.

Systems constructing databases with information associated with a plurality of users, to tasks or tests to be supplied, as well as to the responses of each user to the performance of the tasks, which have been assigned to them are also known.

Some of said proposals describing methods and systems by means of which databases with more or less information regarding different users are created, as well as different ways of using the information of such databases for the purpose that the person responsible for selecting the intervention or interventions, such as a therapist, can evaluate the evolution of a user or a patient while executing one or more tasks, comparing his results with those of other users, or even selecting the tasks to be supplied to the user depending on the results of other users to whom said task has been assigned, are described below.

U.S. Pat. No. 6,964,638 proposes a method for measuring the cognitive capacity of a user, presenting the user with a series of cognitive tests and, among other actions, performing a statistical analysis of the responses to said tests by using information which may include the responses to said tests of other presumably healthy users as reference. The specification of said patent advocates in that the proposal described therein combines the capacity of the statistical analysis with the capacity for collecting data from the responses to cognitive functions.

U.S. Pat. No. 5,711,671 proposes a system including a host computer in connection with several computerized terminals of patients and of therapists. The host has access to a database including both tasks or treatments to be selected by a therapist to assign them to a patient, and the responses to the performance of said tasks by different patients. For one embodiment the host acts as a supervisor for the therapists. Other tasks that the host offers to the therapist are: registering patients on-line, prescribing (and updating) treatments, evaluating clinical progresses, as well as supplying reports. Said patent proposes that the therapist, once having seen the evolution of the patient, prescribes additional tasks or treatment processes to him. It also proposes that the host stores and combines the responses of several patients to enable performing a conductive search for rehabilitation processes.

In addition, U.S. Pat. No. 6,280,198 proposes a method for administering cognitive tests to a patient, remotely monitoring his responses to the tests and evaluating the evolution of his cognitive level based on said responses. Said patent proposes constructing a database which may include cognitive tests, of a reference type or not of a reference type, demographic information of the patient and his responses to the tests.

The specification of U.S. Pat. No. 6,280,198 describes including in the database information of responses to tests for a great number of persons and using this information for selecting proposed therapy programs.

U.S. Pat. No. 6,280,198 also describes performing, depending on its demographic characteristics or on other characteristics, different groups of patients having in common tendencies in their responses and including said groups in the database. Said groups have the purpose of extrapolating the responses of a patient belonging to a group to the rest of members of said group, in order, for example, to select a therapy program for a patient depending on the responses of other patients to similar therapy programs, as well as to evaluate the progress of the patient in comparison with other cases.

U.S. Pat. No. 6,632,174 proposes a method for diagnosing and training cognitive skill of a user for the purpose of selecting one or more tasks to be performed by the user. It proposes storing in a database (local or remote) the responses and historical results of several users to enable performing a cross-validation of the results of a user against a considered acceptable criterion.

U.S. published patent application No. U.S.2014/0235956, whose contents are incorporated by reference, concerns a computerized method for providing a client terminal with an evaluation of a gastrointestinal cancer using a database and comprising several steps involving a comparative treatment of medical data.

None of the foregoing patent documents teach or even suggest the use of the aforementioned database beyond the direct use of said information stored in the database either for performing a statistic analysis, a conductive search, extrapolations or cross-validations of results for different patients. These become insufficient to discover and modulate neural plasticity, because the modification and regulation between existing connections and the establishment of new connections will require not only applying the already known learning strategies but the systematic explorations of new strategies, based on the combination of multifactorial interaction of variables, multivariable evaluation and multivariable monitoring of therapeutic procedures and their interaction with the pre and post administration of tests by traditional/conventional method, monitoring the performance of exercises based on multivariable strategy and verifying the adequacy of any hypothesis by assessing the clinical impact at the end of the process, by conventional testing.

### DESCRIPTION OF THE INVENTION

It is necessary to offer an alternative to the state of the art which allows actually extracting a great profit from the information stored in one of such databases, in relation to a plurality of users or patients, not for simply extracting it directly or for applying statistical criteria on it, but for using it as a raw data source to obtain a net information the classification of which, in different manners, provides a series of candidate predictions related to interventions to be performed or to which the user is to be subjected, based on which one or more final predictions are determined.

The present invention makes up the aforementioned alternative to the state of the art by means of providing, in a first aspect, a method the application of which provides the aforementioned use of the information of one of such databases to achieve the aforementioned objectives of selecting one or more final predictions which allow safely guiding interventions in processes the substrate of which is the neuronal plasticity.

To that end the present invention relates, in a first aspect, to a method for safely guiding interventions in processes the substrate of which is the neuronal plasticity, said interventions including neurorehabilitation, neuroeducation/neurolearning or cognitive neurostimulation, comprising using a central computer server and a plurality of user computer terminals in two way communications with said central computer server, at least a therapist computer terminal and a database server with information regarding a plurality of users, said information being associated to evolutionary variables regarding how such interventions are being performed on said users and responses to performance of said interventions, wherein said information associated to said evolutionary variables includes at least:

values that said evolutionary variables adopt over time, previously, during and posteriorly to said interventions,

values associated to multifactorial variables representing multifactorial interactions of at least said evolutionary variables,

values associated to a multivariable evaluation of at least said evolutionary variables;

values associated to multivariable monitoring of at least said evolutionary variables;

wherein said method comprises by using said computer means and software associated, automatically performing the following steps with said computer means and software associated:

a) generating at least two groups of candidate predictions related to possible interventions, performing at least two steps of classification based on at least heuristic rules on the information of said database including said evolutionary variables considered as a constituent of some basic training data,

b) generating from at least said two groups of candidate predictions a set of training data in meta-level,

c) performing a meta-classification based on at least heuristic rules on said set of training data in meta-level, and

d) determining a group of optimum predictions based on the results of said step c), selecting one of said groups of candidate predictions obtained in step a), or combining said candidate predictions to one another.

For an embodiment, said evolutionary variables are therapeutic variables, said multivariable monitoring of at least said evolutionary variables being related to multivariable monitoring of therapeutic procedures and their interaction with the pre and post administration of tests by traditional/conventional methods, to multivariable monitoring of the performance of tasks based on multivariable strategy, with the purpose of verifying the adequacy of any hypothesis by assessing the clinical impact at the end of the process, by conventional testing.

Said therapeutic variables comprises, for a variant of said embodiment, information related to at least one of the following information contents:

combination of tasks depending on the kind of intervention

combination of strategies within each task

temporal distribution of tasks and of strategies; and

impact that the selected intervention and strategy has in each user, based on its relation with other monitored variables.

For said embodiment where the evolutionary variables are therapeutic variables, said evolutionary variables also comprise information in relation to at least one of:

the cognitive or functional domain where any task is assigned aiming to restore the deficit where the execution of the exercise will have more clinical impact, being a non-exclusive assignation but a profile of relation with different intensity of any exercise to each cognitive or functional domain;

the order of administration of tasks, which implicitly include the order in which any specific deficit is addressed inducing different strategy of recovery by the order in which any recovered function allow the emergence of new functionalities, inducing a continuing evolution of the residual capacity, that need continuing multifactorial modeling by monitoring of any of said multifactorial variables;

specific components of each intervention, defined as any of the elements than will induce a variation in the degree of difficulty to be completed, and that can be modified by the therapist to decrease or increase the difficulty, and allow improving or worsening the performance of the subjects;

the strategy introduced to modify the performance on any subject by means of assigned tasks allowing them to start the recovery of lost functions or the acquisition of new functions from the point where their actual status allow them to engage with the function at the optimal or therapeutic range, wherein said range is defined by the achievement of a minimum percentage of right responses, selected by the therapist, and no more than a percentage of right responses that will indicate proficiency, not recovery or not learning; and

the temporality in that any task and any strategy is suggested based on initial impairment profile, the results and temporal profile of evolutionary variables, and it relationship with final assessment and final achievements.

For the purpose of improving the results to be obtained by means of applying the aforementioned step a), the proposed method comprises, for a preferred embodiment, validating, in step a), the results of said steps of classification from validation data common for validating the results of all the steps of classification, independently and separately from the basic training data, the candidate predictions being performed after said validation.

For an embodiment, the information of said database server also includes information associated to structural variables and functional variables, said multifactorial interactions, multivariable evaluation and multivariable monitoring being related also to said structural and functional variables, together with said evolutionary variables.

The method comprises, in order to obtain better results in said step c), generating in step b) the aforementioned set of training data in meta-level also from said validation data.

For one embodiment, said step a) comprises carrying out said steps of classification independently by means of using two or more classifiers, at least one for each step of classification, differentiated from one another at least in that each of them is based on the application of a respective set of heuristic rules different from that of the other classifier to obtain said two or more groups of candidate predictions different from one another.

Still in said embodiment, once the groups of candidate predictions have been generated, when said step d) comprises selecting one of the groups of candidate predictions, step d) also comprises selecting the classifier and heuristic rules used which have caused said optimum predictions.

For another embodiment of the method proposed by the first aspect of the invention, step a) comprises carrying out said two or more steps of classification by means of using a single classifier based on a single set of heuristic rules, said classifier being used twice or more, once for each step of classification with different input parameters every time to obtain said two or more groups of candidate predictions different from one another, after which for said case when step d) comprises selecting one of the groups of candidate predictions, this also comprises selecting the input parameters of the classifier which have caused said optimum predictions.

The two embodiments described in relation to the way for carrying out the steps of classification of step a) are alternative or complementary, which latter case contemplates performing steps of classification differentiated by using different classifiers, and other steps of classification differentiated in that, although they use one and the same classifier, the latter uses different input parameters every time. For said complementary case, the number of steps of classification is equal or more than three.

For the embodiment in which the steps of classification comprise using different classifiers, the method comprises using classifiers differentiated not only by the heuristic rules to be used but by other characteristics such as: the type of classifier, the operation mode, etc.

In addition, although the steps of classification and of meta-classification performed according to the proposed method are based on at least the application of heuristic rules, the proposed method comprises performing them by using other class of additional rules, such as deterministic rules.

For one embodiment, which will later be referred to as an off-line processing, the method comprises performing steps a) to d) prior to requesting or applying for a prediction in relation to an intervention for a determined user, in which case it comprises, for the purpose of carrying out said prediction, applying the classifier, together with its input parameters and heuristic rules selected after said step d) on data with information regarding said determined user to obtain at least the prediction in relation to an intervention to be performed.

For another alternative embodiment, or an on-line processing, the method comprises performing steps a) to d) after requesting the prediction in relation to an intervention for a determined user, data with information regarding said determined user being included in said database to be used in said step a) to finally obtain at least the prediction in relation to said intervention to be performed.

For the case in which said determined user is a user of the aforementioned plurality of users, the method comprises extracting the data with information regarding said determined user from the database for the purpose of using them in step a) as part of the aforementioned basic training data both for the off-line and on-line processing.

If, in contrast, said determined user is not a user of said plurality of users, i.e., if he is a new user, his data were therefore not incorporated in the database, the method comprises introducing the data with information regarding said determined user in said database both for the on-line and off-line processing, in the latter case the performance of steps a) to d) can be carried out once the data of the new user have already been incorporated in the database or prior to said introduction.

For the purpose of enriching the database and therefore increasing the precision in future predictions by applying the proposed method, the latter comprises:

providing said intervention to said determined user;

subjecting said determined user to said intervention; and

acquiring and recording in said database data with information regarding the results of the determined user been subjected to said intervention and, if appropriate, other new data regarding said determined user for the purpose of updating said database.

The method comprises sequentially performing steps a) to d) again periodically or every time when new data is introduced in said database, the determined predictions, which will be more precise each time, thus being updated over time by the learning caused by executing steps a) to d) again and by updating the information stored in the database.

For an embodiment of the proposed method, the steps of classification for step a) and the step d) of meta-classification are carried out by means of using artificial neural networks, in which case the aforementioned input parameters are related to the parameters of an artificial neural network such as those referring to one or more of the following characteristics: network topology, activation function, end condition, learning mechanism, or to a combination thereof.

In an embodiment complementary to that described in the previous paragraph or independent thereof, the steps of classification for step a) and the step d) of meta-classification are carried out by means of using automatic inductive learning algorithms, carrying out in the step d) the selection of the inductive learning algorithm and/or of its input parameters which have caused the aforementioned optimum predictions.

For said case in which automatic inductive learning algorithms are used for the purpose of carrying out the above so-called off-line processing, the algorithms to be used are of greedy type (such is the case in which artificial neural networks are used).

In contrast, for the case above so-called on line processing, the algorithms to be used are of lazy type, such as those used in inductive methods like reasoning based on cases, the input parameters of which are one or more of the following parameters: indexing type (by sizes, by differences, by similarities, by explanations, etc.), storage type (dynamic memory model (Schank, Lolodner) or category and exemplar model (Porter, Bareiss)), recovery type (closest neighbors, decision trees, SQL type queries screen, etc.) and adaptation type (structural or derivational), or a combination thereof.

The proposed method comprises using any algorithm or strategy known in the field of meta-learning to carry out the different steps of classification described above.

The applications of the proposed method are whichever applications that include processes the substrate of which is the neuronal plasticity, such as those regarding neurorehabilitation, neuroeducation/neurolearning or cognitive neurostimulation, all of them representative of different embodiments of applying the method proposed by the invention.

In terms of the interventions safely guided by means of the proposed method, they comprise, for some embodiments, at least cognitive and/or functional tasks to be performed by the above so-called determined user or subject of said neurorehabilitation, neuroeducation/neurolearning or cognitive neurostimulation.

With regards to the information to be included in the database, the method proposed by the first aspect of the invention comprises including in said step for generating the database information regarding each user of said plurality of users in relation to personal variables (previous to any intervention) and/or structural variables and/or functional variables and/or evolutionary variables comprising therapeutic variables which are defined in more detailed below.

With regards to said personal variables or the bio-psycho-social variables, these refer to all those variables making up the particular background of the life of the user or subject and of his lifestyle.

The present method distinguishes three types of personal variables:

Biological variables: date of birth, age, sex, etc.

Psychological variables: premorbid personality, attitudes of the person, coping styles, etc.

Social variables: place of residence, education level, work status, socioeconomic status, marital status, stability, family support, etc.

The aforementioned structural variables comprise variables which allow defining whether alterations at a structural level exist, as well as for describing the involvement, if it exists, for each of the users, and they comprise one or more of the following variables, and any combination thereof:

Primary and secondary (if any) diagnosis variables.

Variables of etiology (e.g. TBI, stroke, neurodegenerative disease, . . . ).

Variables of lesions in neuroimaging.

Variables of the severity of the lesion.

Variables of time of evolution.

The aforementioned functional variables comprise information in relation to the cognitive aspects of the users assessed by means of a round of neuropyschological examination including one or more of the following variables:

Attention variable which is a complex function formed by specifics sub-processes. The following are distinguished:

1) Sustained attention: allows one to remain alert when facing stimuli for long time periods.

2) Selective attention: capacity for selectively processing information inhibiting other not relevant information.

3) Divided attention: capacity for performing two activities simultaneously.

Language variable represents the capacity of human being to communicate to one another through signs, mainly through linguistic signs, the method distinguishes therein: production, comprehension, nomination, reading, grammar, and pragmatism.

Memory variable defines the cognitive process that allows recording and reproducing information. Memory is not a single function, but rather it can be subdivided based on different classifications like the following:

Time: long and short term memory.

Domain: declarative and implicit memory.

Type of information: verbal or non verbal.

Time phase: encoding, storing and retrieving.

Executive functioning variable is the set of cognitive functions which allow controlling and regulating the conducts directed to an objective or goal, which are integrated by different cognitive capacities, and they include: planning, monitoring, verifying, and inhibiting.

Finally, in terms of the aforementioned evolutionary variables, these comprise information in relation to the success of each user been subjected to one or more interventions, said success being analyzed at least one of the following three levels:

success at level of execution of the cognitive and/or functional task and of the suitability or adequacy of the task proposed for each specific profile of user;

success at level of achievement of the generic objective which is understood as objectified improvements at other cognitive functions in addition to the target function; and

success at level of achievement of long term objective which is understood as a reduction of the functional limitations for the development of daily activities in the case of a neurorehabilitation process, or which is understood as the achievement of a certain degree of neurolearning in the case of a neuroeducation/neurolearning process, or which is understood as an improvement in the stimulated cognitive capacities in the case of cognitive neurostimulation.

The success is also analyzed, for another embodiment, at level of achievement of the immediate objective, which is understood as an improvement in the cognitive function for which the cognitive and/or functional task has been selected.

The evolutionary variables constitute, for some embodiments, new values as a result of development in time of variables selected from the group consisting of biological variables, psychological variables, social variables and any combination thereof.

The higher the number of the variables to be included in the database, the better the result to be obtained, i.e., the final predictions obtained will be more reliable. Therefore, for an embodiment, the method comprises including all the previously described variables in the database and using them as basic training data in step a).

For a preferred embodiment, the method comprises starting said step a) after the prior selection, by the person responsible for selecting the intervention or interventions, of one or more interventions to be applied to a determined user.

With regards to the final predictions determined by means of applying the method proposed by the present invention, for a preferred embodiment these refer to the percentage of success or risk of applying an intervention to a determined user and the method comprises, for a variant of said embodiment, depicting said percentage of success or risk for said determined user by means of the previously described evolutionary variables, and incorporating the new values of the evolutionary variables for said determined user in the database.

If the so-called person responsible for selecting the intervention or interventions is a therapist selecting, for example, a task to be assigned to a patient (a selection that is carried out based on his knowledge on the subject), said therapist requires executing steps a) to d) of the proposed method to be guided in the intervention or task that has been previously selected.

Once steps a) to d) are executed, said guiding supplies him (for example visually through a screen) with a percentage of success or risk of assigning the selected task to the determined user, that percentage allowing the therapist to be guided in the sense of knowing if his selection is considered, by the system implementing the method, as a high or low risk selection, after which the therapist finally decides whether his selection of task is to be maintained or modified.

For the purpose of supplying, for example the therapist, with an additional source of guidance to the aforementioned percentage of success or risk, the method comprises performing a collaborative filtering by means of which integrating the explicit opinion of a plurality of therapists (or of persons responsible for other type of operation when is not the case of carrying out a therapy), by means of, for example, an individual weighting of the determined predictions.

A second aspect of the invention relates to a system for safely guiding interventions in processes the substrate of which is the neuronal plasticity which is suitable for applying the method proposed by the first aspect of the invention and which will be described in more detailed in a subsequent section.

### BRIEF DESCRIPTION OF THE DRAWINGS

The foregoing and other advantages and features will be better understood from the following detailed description of several embodiments in reference to the accompanying drawings, which must be interpreted in an illustrative and non-limiting manner, in which:

FIG. 1 shows a schematic diagram including the different elements taking part in the different steps of the method proposed by the first aspect of the invention for an embodiment; and

FIG. 2 is a schematic depiction of the system proposed by the second aspect of the invention for an embodiment.

### DETAILED DESCRIPTION OF SEVERAL EMBODIMENTS

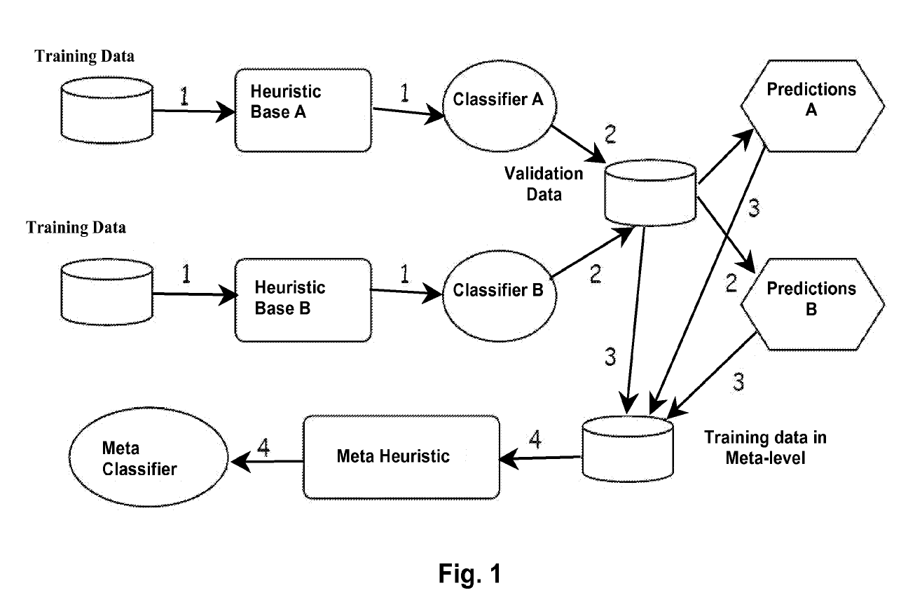

Firstly, referring to FIG. 1 in which, different elements or blocks by means of which steps a) to d) of the method proposed by the first aspect of the invention for an embodiment are implemented, have been depicted.

Specifically the diagram illustrated in FIG. 1 illustrates the previously described embodiment for which step a) comprises carrying out the steps of classification independently by means of using two classifiers indicated in FIG. 1 as “Classifier A” and “Classifier B”, one for each step of classification, differentiated from one another in that each of them is based on the application of a respective set of heuristic rules different from that of the other classifier, indicated in FIG. 1 as “Heuristic Base A” and “Heuristic Base B” to obtain said two groups of candidate predictions different from one another indicated in FIG. 1 as “Predictions A” and “Predictions B”, respectively.

FIG. 1 ultimately depicts a possible meta-learning scenario having the following steps:

1. Classifiers A and B are trained from a set of common training data, heuristic rules or heuristics base A and B, respectively, being applied on them. The sequence is indicated by arrows 1 in FIG. 1.

2. Candidate predictions A and B are generated from the classifiers A and B, respectively, learnt in a set of validation data independent and common for both classifiers. The sequence is indicated by arrows 2 in FIG. 1.

3. A set of training data in the meta-level is generated from the set of validation data and from the candidate predictions A and B generated by the classifiers A and B, respectively, in the set of validation data. The sequence is indicated by arrows 3 in FIG. 1.

4. The final classifier (Meta-classifier) is trained from the set of training data in Meta-level by means of Meta Heuristic using inductive learning in the meta level for integrating the different classifiers A and B, or for improving the performance of each of them independently for the purpose of determining the final or optimum predictions in the previously described step d).

The method proposed by the first aspect of the invention is carried out, for an embodiment, by means of the meta-learning scenario illustrated by FIG. 1, although for other embodiments, unlike the one illustrated, the scenario can be other scenario having greater or lesser complexity.

In terms of the strategies to be applied for implementing the Meta-classifier, these can be strategies of very diverse kinds such as the following: Voting, Weighted Voting or Arbitration for the purpose of obtaining the final prediction in step d) after receiving a request to be classified which, for an embodiment, consists of a task preassigned to a determined patient by a therapist.

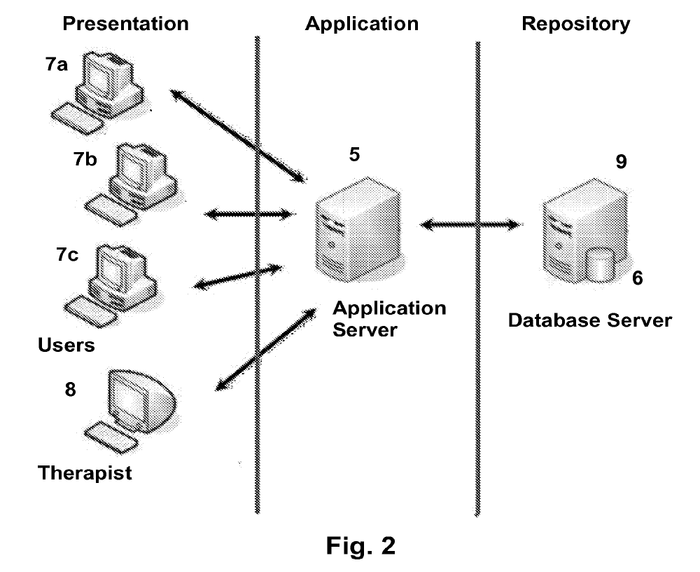

FIG. 2 illustrates an embodiment of the system proposed by the second aspect of the invention which is suitable for applying the method proposed by the first aspect of the invention, and it comprises:

a central computer server 5 with access to a database 6 such as that described above for the method proposed by the first aspect of the invention,

a plurality of computerized user computer terminals 7a, 7b, 7c in two-way communication with said central computer server 5 for receiving, each of them, information in relation to said interventions and for sending the result of the performing of said interventions to the central computer server 5, and

a therapist computer terminal 8 in remote communication with said central computer server 5 for requesting the prediction of an intervention for a determined user, for receiving said requested prediction and for confirming that the information in relation to said intervention, in relation to which said prediction has been required, has been sent by the server to the terminal of said determined user 7a.

The central computer server 5 is provided for carrying out steps a) to d) of the method proposed by the first aspect of the invention.

An architecture of the proposed system divided into three layers has been depicted in said FIG. 2: one presentation layer including the different terminals 7a-7c and 8, one application layer including the aforementioned central server 5 referred to as application server and one repository layer, further including, other than the aforementioned database 6, a database server 9 through which the server 5 accesses the database 6.

An embodiment based on the architecture of the system illustrated in FIG. 2, which is also referred to as a platform, the operation of which is described below, is explained next.

Users can execute the tasks, which have been programmed by the therapist regardless of their physical location through this platform, with the only requirements of having an Internet connection and the client software application installed in the computer or terminal of the user 7a-7c.

The platform server 5 provides a remote access web interface where the client software connects for the purpose of authenticating it, retrieving the information on the tasks to be performed in the current session and transmitting the generated results to the database server 9.

It therefore works based on an architecture structured in three independent layers that interact with one another:

### The Presentation Layer

This layer joins all the aspects of the software that have to do with the interfaces and the interaction of the system with the users and therapists. The client software is installed in each of the computers, 7a-7c and 8, accessing the platform through the interface provided in this layer such that the therapists can provide criteria for the tasks to be executed by its users and these users executing them regardless of their physical location. This communication is performed by means of XML-RPC Web Services, which also operates through the HTTP or HTTPS protocol which a priori assures that the communications will not be blocked by routers or firewalls unless these have explicitly disabled the transmissions through ports 80 or 443. Since this protocol is executed over the TCP transport protocol, all the data sent will be received by the recipient.

### The Application Layer

Requests generated from the presentation layer by the client software are received and managed in this layer and the results are displayed. It interacts with the repository layer for requesting the storage or retrieval of data from the database server 9. In general terms, it is referred to as the layer where the logic of the method concentrates, i.e., the rules governing the behaviour at the operational level of the application, for the purpose of carrying out the method proposed by the first aspect of the invention for sending a final prediction to the therapist terminal 8, located in the presentation layer, after the therapist requested it and for sending the tasks assigned by the therapist to the user terminals 7a-7c.

### The Repository Layer

This layer gathers all the aspects of the software that have to do with the management of the persistent data, managing them in a transparent manner at the application layer.

Some of the elements used based on the architecture structured in three layers illustrated by FIG. 2 are subsequently broken down. These are:

Design pattern: By taking into account this encapsulation of the system in these three independent levels or layers, the use of design pattern, Model-View-Controller (MVC), is proposed. This design pattern explicitly separates the data access and the logic of the method for presenting the data and the interactions with the users and therapists by means of introducing an intermediate component: the controller. For this purpose, the platform J2EE is used, where each of the three components of the design pattern will have the following functionality:

Model: Any access to the databases 6 will use some of the functions provided by the classes of the Model. These classes are known as Data access Object (DAO) and they are applied from the classes known as Action. The classes, which correspond to a representation of a table of a database, are the Value Object (VO) detailed below.

View: It corresponds to the web interface which is seen by the users and therapists and with which they must interact. It is implemented by using Java Server Pages (JSP) with HTML and CSS code.

Controller: It is a Servlet which receives the requests from View and which directs them to the corresponding Model.

Every web application in the Apache Tomcat server, which is the one used in the system for an embodiment, contains two non-public directories of information in relation to the execution of the web. These directories are:

META-INF: It contains the file manifest.mf with generic information on the application and the file context.xml which defines the context with resources used by the web, such as for example the access to database 6.

WEB-INF: It contains the compiled classes, libraries and the file web.xml defining the structure of the application with the existing servlets, redirections and maps. It also contains some so-called property files, which are the following:

actions.properties: indicates for each entity which is the file with the view (JSP) and which is the file with the action (Action).

params.properties: defines the general parameters of the application.

The process followed by the system serving as a webpage is described below:

1. A request is generated from a URL direction with the format protocolo://server:puerto/peticion.do. Since the directions ended in .do are mapped to the controller, as has been defined in the file web.xml, the latter will receive the request.

2. The controller will pass the captured request to the next action, since it is loaded with the file action properties, the action that must be executed is known.

3. The action corresponding to the request receives the data from the former and provides the corresponding queries to the database 6 through the DAO corresponding to the entities from which data must be retrieved, inserted, deleted or updated.

4. When the action ends, the controller calls the view corresponding to the request.

5. The view (JSP) receives the data returned by the action, encapsulated in the variable of response type. The view contains Java code that processes the received data (collecting VO, etc.), compiles them in execution time and sends the resulting HTML code to the web navigator.

The Model of the design pattern is therefore made up of Entities that are J2EE components representing the data stored in the database. Each entity corresponds to one of the following three objects:

Data access Object: implements the class containing all the methods for providing queries to the database; these queries allow retrieving, inserting, deleting and updating data.

Value Object: these are objects used for transferring information between processes and have no other behavior than to store and retrieve their own data.

Primary Key: This object is a class, which stores the primary key of the entity. This type of primary key is defined in this class as the one, which allows detaching it in all the platform.

Some of the entities implemented in the platform are presented below:

User: Manages all data related to the users of the application regardless of the role of each of them (users, therapists, . . . ).

Language: Serves to manage the languages (Spanish, Catalan, . . . ) contemplated by the platform and allows aggregating new ones.

Function: It is used to save the information relating to supported functions (cognitive, . . . ) and sub-functions.

Task: It contains the information in relation to the tasks proposed by the therapists to the users.

Parameters: It contains the information of the input and output parameters of a given task.

Session: It contains the data related to sessions of tasks

The planning of tasks and the query of results have the peculiarity that an action on some of the elements of the page does not cause the page to be completely refreshed but rather only those parts containing new information are refreshed. This is achieved by using a group of interrelated web development techniques known as AJAX (Asynchronous JavaScript and XML). This technology allows performing asynchronous calls in JavaScript to a web server by using the object XMLHttpRequest, the response thereof is also processed in an asynchronous manner for dynamically changing the outlook of the webpage. This provides a greater interactivity, functionality, efficiency and ease of use of the webpages.

On the application server 5 side three servlets, which are those that receive these asynchronous requests for the planning of tasks section, have been created. These servlets are:

BlockServlet: It manages the blocks of tasks and the tasks within them, it also allows changing day in the calendar, loading the plan on the selected day.

SchedulerServlet: It navigates the tree of tasks generated by grouping them into functions and sub-functions.

With regard to the query of results, the following servlet is used:

ResultServlet: it allows navigation by a calendar, loading the information in relation to the results of the selected session for each selected day.

On the client side, i.e., the navigator, the files ajax.js for planning and query of results have been created. These files contain all the functions that are used for communicating in an asynchronous manner with the server 5. In order to avoid the implementation of the HTTP calls, a free code library known as SACK, which provides an API, which facilitates calling the server as well as obtaining the response, is used.

Regarding to the responses received by the server 5, they can be in plain text format or in the XML format. In the first case, it is read directly, if it is of XML format the XML DOM functions of JavaScript are used to read the received data. In both cases, in order to dynamically modify the display of the webpage the JavaScript functions known as HTML DOM are used.

As mentioned above, the system allows that the users to not be physically located in any particular location (hospital, care center, etc.) to perform the tasks assigned by the therapists after they have been conveniently guided for measuring the final prediction determined according to step d) of the method proposed by the first aspect of the invention. The technology selected for supporting this performance are the XML-RPC web services due to its simplicity, minimalism and ease of use. The data are sent in XML format and the conversions between remote calls and XML are performed by the libraries in a transparent manner to the programmer. It further allows abstracting the web application and the client software from the programming language used.

The extensive and specific description of the previous embodiment has demonstrated that the system proposed by the second aspect of the invention has been implemented in practice to demonstrate the suitability thereof and of the method proposed by the first aspect of the invention applied by the system.

### An Embodiment of the Invention Encompasses

(i) the interventions on which a prediction is requested involve cognitive training and rehabilitation of an impaired user.

(ii) the stored information in the database and that is used in the method to include data results of validated psychometric tests, which should be considered as objective indicators of neurobiological functions, performed on a plurality of users when executing cognitive and/or functional tasks. This means that the method operates with data results, i.e. numerical values obtained from cited psychometric tests.

(iii) the data results that indicate a degree of recovery of lost functions or acquisition of new functions by any of the users in said database.

(Iv) the determination of a group of optimal predictions from the data results by using a processor of the central computer server and by using a specific algorithm and machine learning techniques. That is, referring to a percentage of success or risk of applying a selected intervention to the impaired user.

The method includes features of receiving from a database data results, which are objective indicators of neurobiological functions, applying a specific algorithm and machine learning techniques and outputting as a result a group of determined predictions. The features define how the use of the central computer server contributes to the invention more than in a nominal or insignificant manner.

Moreover, the method provides an improvement in computer related technology as it is advantageous to be able for guiding interventions in processes the substrate of which is the neural plasticity in an efficient manner to improve an underlying technical process within the technical field of machine learning and improve accuracy (proximity to a best prediction) of the candidate predictions. In particular the steps of the method provide better results and allow optimizing the resources in the given context. The steps including

a) generating at least two groups of candidate predictions related to possible interventions on said impaired user, by performing at least two steps of classification based on at least heuristic rules on the extracted information of said database including said evolutionary variables considered as a constituent of some basic cognitive training data,

b) generating from at least said two groups of candidate predictions a set of training data in meta-level,

c) performing a meta-classification based on at least heuristic rules on said set of training data in meta-level, and

d) determining a group of optimum predictions based on the results of said step c), selecting one of said groups of candidate predictions obtained in step a), or combining said candidate predictions to one another.

A further embodiment is for a computerized method for safely guiding interventions for a user in processes the substrate of which is the neuronal plasticity. The interventions include cognitive training and rehabilitation of an impaired user. The method comprises the steps of initially receiving, extracting, performing, supplying, sending and further receiving.

The initially receiving step entails receiving, by a central computer server associated with a database, a request for a prediction of an intervention for the impaired user and data about the impaired user from a therapist computer terminal in remote communication with the central computer server.

The extracting step entails extracting, by the central computer server, database information upon reception of the request. The database information is that stored in the database from a plurality of users having a similar impairment profile. The information includes data results of validated psychometric tests, as objective indicators of neurobiological functions performed on the users when executing cognitive and/or functional tasks, and being associated with evolutionary variables regarding how such interventions are being performed on the plurality of users and responses of the plurality of users to performance of the interventions indicating a recovery of lost functions or acquisition of new functions. The data results include measures concerning reliability, stability, internal consistency, equivalence and validity of the psychometric tests.

The information associated with said evolutionary variables includes at least values that:

the evolutionary variables adopt over time, previously, during and posteriorly to said interventions,

are associated with multifactorial variables representing multifactorial interactions of at least said evolutionary variables,

are associated with a multivariable evaluation of at least said evolutionary variables;

are associated with multivariable monitoring of at least said evolutionary variables.

The performing step entails automatically performing, by a processor of the central computer server, the following steps, using the extracted information including the data results:

a) generating at least two groups of candidate predictions related to possible interventions on said impaired user, by performing at least two steps of classification based on at least heuristic rules on the extracted information of said database including said evolutionary variables considered as a constituent of some basic cognitive training data,

b) generating from at least said two groups of candidate predictions a set of training data in meta-level,

c) performing a meta-classification based on at least heuristic rules on said set of training data in meta-level, and

d) determining a group of optimum predictions based on the results of said step c), selecting one of said groups of candidate predictions obtained in step a), or combining said candidate predictions to one another.

The group of optimum predictions refer to a percentage of success or risk of applying an intervention to the impaired user. The percentage of success or risk for the impaired user being depicted by means of said evolutionary variables and incorporating new values of the evolutionary variables for the impaired user in the database.

The supplying step includes supplying, by the central computer server to the therapist computer terminal, the group of determined optimal predictions, in order to make a decision as to whether maintaining or modifying the interventions contained in the optimal predictions.

The step of sending includes sending, by the central computer server to the impaired user via a user computer terminal in bidirectional communication with the central computer server an intervention programmed by the therapist of the group of optimal predictions based on said decision taken by the therapist computer server.

The step of further receiving includes further receiving, by the central computer server, results of the programmed intervention performed by the impaired user and including said data results in the database.

A person skilled in the art could introduce changes and modifications in the described embodiments without departing from the scope of the invention as defined in the attached claims.

References for test procedures and validation procedure for validated psychometric tests.

[1]. Cook D A, Beckman T J. Current concepts in validity and reliability for psychometric instruments: theory and application. Am J Med. 2006 February;119(2):166.

[2]. Pittman J, Bakas T. Measurement and instrument design. J Wound Ostomy Continence Nurs. 2010 November December;37(6):603-7.

[3]. Terwee C B, Bot S D, Boer M R, van der Windt, Knol D L, Dekker J, et al. Quality criteria were proposed for measurement properties of health status questionnaires. J Clin Epidemiol. 2007 January;60(1):34-42.

[4] Salmond S S. Evaluating the reliability and validity of measurement instruments. Orthop Nurs. 2008 January February;27(1):28-30.

[5] Vet H C, Terwee C B, Knol D L, Bouter L M. When to use agreement versus reliability measures. J Clin Epidemiol. 2006 October;59(10):1033-9.

[6]. Keszei A P, Novak M, Streiner D L. Introduction to health measurement scales. J Psychosom Res. 2010 April;68(4):319-23.

[7]. Streiner D L. Starting at the beginning: an introduction to coefficient alpha and internal consistency. J Pers Assess. 2003 February;80(1):99-103.

[8] Streiner D L, Kottner J. Recommendations for reporting the results of studies of instrument and scale development and testing. J Adv Nurs. 2014 September;70(9):1970-9.

[9] Cortina J M. What is coefficient alpha? An examination of theory and applications. J Appl Psychol. 1993;78(1):98-104.

[10] Aaronson N, Alonso J, Burnam A, Lohr K N, Patrick D L, Perrin E, et al. Assessing health status and quality-of-life instruments: attributes and review criteria. Qual Life Res. 2002 May;11(3):193-205.

[11] Heale R, Twycross A. Validity and reliability in quantitative studies. Evid Based Nurs. 2015 July;18(3):66-7.

[12] Keszei A P, Novak M, Streiner D L. Introduction to health measurement scales. J Psychosom Res. 2010 April;68(4):319-23.

[13] Polit D F, Beck C T. Fundamentos de pesquisa em enfermagem: métodos, avaliação e utilização. 7 ed. Porto Alegre: Artmed; 2011.

[14] Salmond S S. Evaluating the Reliability and Validity of Measurement Instruments. Orthop Nurs. 2008 January-February;27(1):28-30.

[15] Roberts P, Priest H. Reliability and validity in research. Nurs Stand. 2006 July;20(44):41-5.

[16] Kimberlin C L, Winterstein A G. Validity and reliability of measurement instruments used in research. Am J Health Syst Pharm. 2008 December;65(23):2276-84.

[17] Polit D F. Assessing measurement in health: beyond reliability and validity. Int J Nurs Stud. 2015 November;52(11):1746-53.

[18] Kimberlin C L, Winterstein A G. Validity and reliability of measurement instruments used in research. Am J Health Syst Pharm. 2008 December;65(23):2276-84.

[19] Coluci M Z O, Alexandre N M C, Milani D. Construção de instrumentos de medida na área da saúde. Cienc Saude Coletiva. 2015 march;20(3):925-36.

[20] Polit D F, Beck C T. The content validity index: are you know what's being reported? Critique and recommendations. Res Nurs Health. 2006 October;29(5): 489-97.

[21] Kimberlin C L, Winterstein A G. Validity and reliability of measurement instruments used in research. Am J Health Syst Pharm. 2008 December;65(23):2276-84.

[22] Hair Junior J F, Black W C, Babin B J, Anderson R E, Tathan R L. Análise multivariada de dados. 6 ed. Porto Alegre: Bookman; 2009.

[23] Chin W W, Newsted P R. Structural equation modelling analysis with small samples using partial least squares. In.: Hoyle R H. Statistical strategies for small sample research. Thousand Oaks, Calif.: Sage; 1999. p. 307-41.

[24] Fuster J M. The reticular paradigm of cortical memory. Rev Neurol. 2010 Mar. 3;50 Suppl 3:S3-10.

[25] Rizzolatti G, Fogassi L. The mirror mechanism: recent findings and perspectives. Philos Trans R Soc Lond B Biol Sci. 2014 Apr. 28;369(1644):20130420.

[26] Grafton S T. Embodied cognition and the simulation of action to understand others. Ann N Y Acad Sci. 2009 March;1156:97-117.

### ANNEX

### Neuropsychological Tests

1. Attention:

1.1 Sustained Attention, Divided Attention

1.1.1 Continuous Performance Task Test (Conners & Sitarenios, 2011)

The Conners Continuous Performance Test 3rd Edition is a task-oriented computerized assessment of attention-related problems in individuals aged 8 years and older. By indexing the respondent's performance in areas of inattentiveness, impulsivity, sustained attention, and vigilance, the Conners CPT 3 can be useful to the process of diagnosing Attention-Deficit/Hyperactive Disorder (ADHD) and other neurological conditions related to attention.

Test procedure: during the 14-minute, 360-trial administration, respondents are required to press the spacebar or wired mouse button when any letter except “X” appears. Once complete, the computer generates two easy-to-use reports that better guide assessors through each step of the recommended interpretation process.

Validation procedure and internal consistency: one measure of a test's internal consistency is split-half reliability, which has been previously used to establish the reliability of other continuous performance tests. Split-half reliability estimates of the Conners CPT 3 scales were calculated for the normative and clinical samples. Results were very strong across all scores, the median split-half reliability estimate was 0.92 for the norm samples and 0.94 for the clinical samples (all correlations were significant, p<0.001). These results indicate that the Conners CPT 3 demonstrates excellent internal consistency for both the normative and clinical groups. (Conners & Sitarenios, 2011).

Test-retest reliability: refers to the consistency of scores obtained from the same respondent on separate occasions over a specified period of time. To estimate the test-retest reliability of the Conners CPT 3, a sample of 120 respondents from the general population completed the Conners CPT 3 twice with a 1- to 5-week interval between administrations. The median test-retest correlation was 0.67. These results suggest a good level of test-retest reliability. (Conners & Sitarenios, 2011).

1.1.2 Trail Making Test-A (Reitan & Wolfson, 1993)

The Trail Making Test is a neuropsychological test of visual attention and task switching. It consists of two parts in which the subject is instructed to connect a set of 25 dots as quickly as possible while still maintaining accuracy.[1] The test can provide information about visual search speed, scanning, speed of processing, mental flexibility, as well as executive functioning.[1]

Test procedure: requires the subject to connect a sequence of 25 consecutive targets on a sheet of paper or computer screen, in a similar manner to a child's connect-the-dots puzzle. There are two parts to the test: in the first, the targets are all numbers (1, 2, 3, etc.) and the test taker needs to connect them in sequential order; in the second part, the subject alternates between numbers and letters (1, A, 2, B, etc.).(Bowie 2006) If the subject makes an error, the test administrator corrects them before the subject moves on to the next dot (Bowie 2006)

The goal of the test is for the subject is to finish both parts as quickly as possible, with the time taken to complete the test being used as the primary performance metric. The error rate is not recorded in the paper and pencil version of the test, however, it is assumed that if errors are made it will be reflected in the completion time (Tombaugh, 2004). The second part of the test, in which the subject alternates between numbers and letters, is used to examine executive functioning (Tombaugh, 2004).

1.2 Selective Attention

1.2.1. Wechsler Adult Intelligence Scale (Wechsler, 1997)

WAIS is one of the most important tests for the clinical evaluation of the intellectual capacity of adults aged 16 to 89 years.

Test procedure: is made up of 14 sub-tests, in which vocabulary, similarity, arithmetic, digit span, information, comprehension and letter-number sequencing belong to the verbal IQ scale, and picture completion, digit-symbol coding, block design, matrix reasoning, picture arrangement, symbol search and object assembly are part of the performance IQ scale. The full scale IQ score is obtained by adding the verbal and performance IQ scores. In addition to these measures, it is also possible to calculate indices for verbal comprehension, perceptual organization, working memory and processing speed.

2. Memory:

2.1 Visual and Verbal Memory:

2.1.1 The Rey Auditory Verbal Learning Test (Rey, 1964)

Evaluates a wide diversity of functions: short-term auditory-verbal memory, rate of learning, learning strategies, retroactive, and proactive interference, presence of confabulation of confusion in memory processes, retention of information, and differences between learning and retrieval.

Test procedure; Participants are given a list of 15 unrelated words repeated over five different trials and are asked to repeat. Another list of 15 unrelated words are given and the client must again repeat the original list of 15 words and then again after 30 minutes. Approximately 10 to 15 minutes is required for the procedure (not including 30 min. interval).

Validation procedure: See Lezak et al. (2004) and Strauss et al. (2006) for reliability and validity information. Most of the following details of psychometric properties are provided by the NINDS TBI CDE project.

Test-retest reliability: is good for total recall over 5 trials, 0.60-0.70 over one year (Mitrushina & Satz, 1991). A recent Australian study indicated poor test-retest reliability over 1 year (0.26 to 0.64) in a cohort of normal 18-34 year olds for individual trials (total recall was 0.60). This was consistent with Geffen et al. (1994) following an interval of 6-14 days and the 1-year test-retest reliability of 0.55 reported by Snow, Tierney, Zorzitto, Fisher, and Reid (1988).

Internal reliability: of the total score is high (alpha coefficients>0.90) (van den Burg & Kingma, 1999). Extensive literature regarding good validity including construct, criterion and predictive. For specific information refer to Strauss et al. (2006).

3. Executive Functions:

3.1 Inhibition:

3.1.1 Stroop Test (Golden, 1994)

Test procedure: There are different test variants commonly used in clinical settings, with differences between them in the number of subtasks, type and number of stimulus, times for the task, or scoring procedures. (Howieson, 2004). All versions have at least two numbers of subtasks. In the first trial, the written color name differs from the color ink it is printed in, and the participant must say the written word. In the second trial, the participant must name the ink color instead.

However, there can be up to four different subtasks, adding in some cases stimuli consisting of groups of letters “X” or dots printed in a given color with the participant having to say the color of the ink; or names of colors printed in black ink that have to be read (Howieson, 2004). The number of stimuli varies between fewer than twenty items to more than 150, being closely related to the scoring system used. While in some test variants the score is the number of items from a subtask read in a given time, in others it is the time that it took to complete each of the trials (Howieson, 2004). The number of errors and different derived punctuations are also taken into account in some versions (Howieson, 2004).

3.2 Flexibility, Categorization

3.2.1 Wisconsin Card Sorting Test (Heaton, Chelune, Talley, Kay, & Curtiss, 1997)

This is a popular neuropsychological measure used to assess concept formation, abstract reasoning, and the ability to shift cognitive strategies in response to changing environmental contingencies.

Test procedure: People have to classify cards according to different criteria. There are four different ways to classify each card, and the only feedback is whether the classification is correct or not. One can classify cards according to the color of its symbols, the shape of the symbols, or the number of the shapes on each card. The classification rule changes every 10 cards, and this implies that once the participant has figured out the rule, the participant will start making one or more mistakes when the rule changes. The task measures how well people can adapt to the changing rules.

3.2.2 Letter Fluency Test (Artiola i Fortuny, Hermosillo Romo, Heaton, & Pardee III, 1999)

Test procedure: Phonemic verbal fluency (PVF) The evaluated individual must produce words that begin with certain letters, frequently F-A-S (PVF FAS). They cannot use either derivatives of the same word or first names. Although the letters F-A-S are also used among Spanish speakers, there are some authors, (Artiola i Fortuny, 1999) who have proposed the use of other letters (P, M, and R) in order to minimize the effects of language. Nevertheless, the use of these letters is less common (Casals-Coll, 2013)

### REFERENCES FOR ANNEX

(Rey, 1964) L'examen clinique en psychologie. Paris: Presses universitaires de France.

(Conners & Sitarenios, 2011) Conners' Continuous Performance Test. Encyclopedia of Clinical Neuropsychology, 681-683.

(Reitan & Wolfson, 1993) The Halstead-Reitan neuropsychological test battery: Theory and clinical interpretation (2nd ed.). Neuropsychology Press.

(Wechsler, 1997) Manual for the Wechsler Adult Intelligence Scale-Third Edition (WAIS-III). San Antonio, Tex.: Psychological Corporation.

(Golden, 1994) STROOP Test de colores y palabras. Madrid: TEA.

(Heaton, Chelune, Talley, Kay, & Curtiss, 1997) Heaton, R. K., Chelune, G. J., Talley, J. L., Kay, G. G., & Curtiss, G. (1997). WCST: Test de clasificación de tarjetas Wisconsin. Madrid: TEA.

(Artiola i Fortuny, Hermosillo Romo, Heaton, & Pardee III, 1999) Artiola i Fortuny, L., Hermosillo Romo, D., Heaton, R. K., & Pardee III, R. E. (1999). Manual de normas y procedimientos para la batería neuropsicológica en Español. Tucson: mPress.

Snow, W., Tierney, M., Zorzitto, M., Fisher, R., & Reid, D. (1988). One-year test-retest reliability of selected neuropsychological tests in older adults. Journal of Clinical and Experimental Neuropsychology, 10(60).

Mitrushina, M., Satz, P., Chervinsky, A., & D'Elia, L. (1991). Performance of four age groups of normalelderly on the rey Auditory-Verbal learning test. Journal of clinical psychology, 47(3), 351-357.

Geffen, G. M., Butterworth, P., & Geffen, L. B. (1994). Test-retest reliability of a new form of the auditory verbal learning test (AVLT). Archives of Clinical Neuropsychology, 9(4), 303-316.

van den Burg, W., & Kingma, A. (1999). Performance of 225 Dutch school children on Rey's Auditory Verbal Learning Test (AVLT): parallel test-retest reliabilities with an interval of 3 months and normative data. Archives of Clinical Neuropsychology, 14(6), 545-559.

Lezak, M. D. (2004). Neuropsychological asessment 4 Ed: Oxford university press.

Strauss, E. H. (2006). A compendium of neuropsychological tests: Administration, norms, and commentary: Oxford University Press.

(Bowie 2006) Bowie, C.R.C.R; P.D.P.D Harvey (2006). “Administration and interpretation of the trail making test”. Nature Protocols. 1 (5): 2277-2281. doi:10.1038/nprot.2006.390.

Tombaugh, (2004). “Trail Making test A and B: Normative Data Stratified by Age and Education”. Archives of Clinical Neuropsychology. 19 (2): 203-214. doi:10.1016/s0887-6177(03)00039-8. PMID 15010086.

Howieson, Diane Black; Lezak, Muriel Deutsch; Loring, David W. (2004). “Orientation and attention”. Neuropsychological assessment. Oxford [Oxfordshire]: Oxford University Press. pp. 3365-367. ISBN 0-19-511121-4. Retrieved 2009-03-06.

Casals-Coll M, Sánchez-Benavides G, Quintana M, et al. Estudios normativos españoles en población adulta joven (proyecto NEURONORMA jóvenes): normas para los test de fluencia verbal [Spanish normative studies in young adults (NEURONORMA young adults project): norms for verbal fluency tests]. Neurología 2013; 28:33-40

What is claimed is:

1. A computerized method for safely guiding interventions for a user in processes the substrate of which is the neuronal plasticity, said interventions including cognitive training and rehabilitation of an impaired user, wherein the method comprises
receiving, by a central computer server associated with a database, request for a prediction of an intervention for said impaired user and data about the impaired user from a therapist computer terminal in remote communication with the central computer server;
extracting, by the central computer server, database information upon reception of said request, said database information being that stored in the database from a plurality of users having a similar impairment profile, wherein said database information includes data results of validated psychometric tests, as objective indicators of neurobiological functions performed on said users when executing cognitive and/or functional tasks, and being associated with evolutionary variables regarding how such interventions are being performed on said plurality of users and responses of said plurality of users to performance of said interventions indicating a recovery of lost functions or acquisition of new functions, said data results including measures concerning reliability, stability, internal consistency, equivalence and validity of the psychometric tests, wherein said information associated to said evolutionary variables includes at least:
values that said evolutionary variables adopt over time, previously, during and posteriorly to said interventions,
values associated with multifactorial variables representing multifactorial interactions of at least said evolutionary variables,
values associated with a multivariable evaluation of at least said evolutionary variables;
values associated with multivariable monitoring of at least said evolutionary variables;
automatically performing, by a processor of the central computer server, following steps, using the extracted information including said data results:
a) generating at least two groups of candidate predictions related to possible interventions on said impaired user, by performing at least two steps of classification based on at least heuristic rules on the extracted information of said database including said evolutionary variables considered as a constituent of some basic cognitive training data,
b) generating from at least said two groups of candidate predictions a set of training data in meta-level,
c) performing a meta-classification based on at least heuristic rules on said set of training data in meta-level, and
d) determining a group of optimum predictions based on the results of said step c), selecting one of said groups of candidate predictions obtained in step a), or combining said candidate predictions to one another,
wherein said group of optimum predictions refer to a percentage of success or risk of applying an intervention to the impaired user, said percentage of success or risk for said impaired user being depicted by means of said evolutionary variables and incorporating new values of the evolutionary variables for said impaired user in the database;
supplying, by the central computer server to the therapist computer terminal the group of determined optimal predictions, in order to make a decision as to whether maintaining or modifying the interventions contained in the optimal predictions;
sending, by the central computer server to the impaired user via a user computer terminal in bidirectional communication with the central computer server, an intervention programmed by the therapist of said group of optimal predictions based on said decision taken by the therapist computer server; and
receiving, by the central computer server, results of the programmed intervention performed by the impaired user and including said data results in said database.

2. The method of claim 1, wherein said evolutionary variables are therapeutic variables, said multivariable monitoring of at least said evolutionary variables being related to multivariable monitoring of therapeutic procedures and their interaction with pre and post administration of tests by traditional/conventional methods, to multivariable monitoring of the performance of tasks based on multivariable strategy, with a purpose of verifying adequacy of any hypothesis by assessing the clinical impact at an end of the process, by conventional testing.

3. The method of claim 1, wherein said information of said database server also includes information associated to structural variables and functional variables, said multifactorial interactions, multivariable evaluation and multivariable monitoring being related also to said structural and functional variables, together with said evolutionary variables.

4. The method of claim 1, wherein said evolutionary variables also comprise information in relation to success of each impaired user who has been subjected to one or more interventions, said success being analyzed at least in one of the following three levels:
at a level of execution of the cognitive and/or functional task and of the suitability or adequacy of the task proposed for each specific profile of user;
at a level of achievement of generic objective, which is an objectified improvement at other cognitive functions in addition to a target function; and
at a level of achievement of long term objective, which is a reduction of functional limitations for development of daily activities in case of a neurorehabilitation process, or which is achievement of a certain degree of neurolearning in case of a neuroeducation/neurolearning process, or which is understood as an improvement in stimulated cognitive capacities in case of cognitive neurostimulation.

5. The method of claim 3, wherein said success is also analyzed at a level of achievement of an immediate objective, which is an improvement in cognitive function for which the cognitive and/or functional task has been selected.

6. The method of claim 2, wherein said evolutionary variables also comprise information in relation to each of cognitive or functional domain where any task is assigned aiming to restore deficit where execution of exercise will have more clinical impact, being a non-exclusive assignation but a profile of relation with different intensity of any of the exercise to each of the cognitive or functional domain.

7. The method according to claim 1, wherein said step a) comprises carrying out said at least two steps of classification independently, by means of using two respective classifiers differentiated from one another, at least in that each of them is based on applying a respective set of heuristic rules different from that of the other classifier to obtain said at least two groups of candidate predictions which are different from one another.

8. The method according to claim 7, wherein when said step d) comprises selecting one of said groups of candidate predictions, step d) also comprises selecting the classifier and heuristic rules used which have caused said optimum predictions.

9. The method according to claim 8, wherein said step a) comprises carrying out said at least two steps of classification by means of using a single classifier based on a single set of heuristic rules, said classifier being used at least twice, once for each step of classification with different input parameters every time.

10. The method according to claim 9, wherein said step d) comprises selecting one of said groups of candidate predictions, and said step d) also comprises selecting the input parameters of said classifier which have caused said optimum predictions.

11. The method according to claim 1, wherein:
if said impaired user is a user of said plurality of users, the method comprises extracting said data with information regarding said user from said database; or
if said impaired user is not a user of said plurality of users, the method comprises introducing said data with information regarding said impaired user in said database.

12. The method according to claim 11, comprising:
providing said intervention to said impaired user r;
subjecting said impaired user to said intervention; and
acquiring and recording in said database data with information regarding the results of the impaired user being subjected to said intervention and, if appropriate, other new data regarding said impaired user for the purpose of updating said database.

13. The method according to claim 1, wherein it comprises sequentially performing said steps a) to d) again periodically or every time new data is introduced in said database.

14. The method according to claim 1, wherein said step a) comprises validating the results of said steps of classification from validation data common for validating the results of all the steps of classification, the candidate predictions being performed after said validation.

15. The method according to claim 14, wherein said step b) comprises generating said set of training data in meta-level also from said validation data.

16. The method according to claim 10, wherein said steps of classification of said step a) and said step c) of meta-classification are carried out by means of using artificial neural networks, said input parameters being at least related to one of the following characteristics of an artificial neural network: network topology, activation function, end condition, learning mechanism, or to a combination thereof.

17. The method according to claim 1, wherein said steps of classification of said step a) and said step c) of meta-classification are carried out by means of using automatic inductive learning algorithms, carrying out in said step d) the selection of the inductive learning algorithm and/or of its input parameters which have caused the aforementioned optimum predictions.

18. The method according to claim 1, wherein said evolutionary variables constitute new values as a result of development in time of variables selected from the group consisting of biological variables, psychological variables, social variables and any combination thereof.

19. The method according to claim 3, wherein said structural variables comprise variables which allow defining whether alterations at a structural level exist, as well as describing an involvement, if it exists, for each of the users, and the structural variables comprise further variables selected from the group consisting of primary and secondary diagnosis variables, if appropriate, variables of etiology, variables of lesions in neuroimaging, variables of the severity of the lesion, variables of time of evolution and any combination thereof.

20. The method according to claim 3, wherein said functional variables comprise information in relation to the cognitive aspects of the users assessed by means of a round of neuropsychological examination, and they comprise additional variables selected from the group consisting of attention variables, language variables, memory variables, executive functioning variables and any combination thereof.
